(19) United States
(12) Patent Application Publication
(10) Pub. No.: US 2025/0057468 A1
(43) Pub. Date: Feb. 20, 2025

(54) SKIN PATCH FOR DETERMINATION OF SWEATING LOCATION AND RATE

(71) Applicant: Clutch, Inc. DBA Carpe, Durham, NC (US)

(72) Inventors: Kasper Kubica, Durham, NC (US); David Spratte, Durham, NC (US); Laken Williams, Raleigh, NC (US)

(21) Appl. No.: 18/769,930

(22) Filed: Jul. 11, 2024

Related U.S. Application Data
(60) Provisional application No. 63/519,890, filed on Aug. 16, 2023.

(51) Int. Cl. A61B 5/00

(52) U.S. Cl. CPC A61B 5/4266; A61B 5/6833

(57) ABSTRACT

Provided herein are systems, methods and compositions for a Skin Patch for Determination of Sweating Location and Rate. A method of manufacturing the skin patch is disclosed and comprises a process of applying a reactive chemical mixture which changes color in the presence of moisture.

### CROSS-REFERENCE TO RELATED APPLICATIONS

This application claims priority to U.S. provisional application Ser. No. 63/519,890, filed Aug. 16, 2023, herein incorporated by reference in its entirety.

### BACKGROUND

This invention relates to the visual identification of localization and intensity of phenomena occurring on certain parts of the skin which may not otherwise be easily visually identifiable, and more specifically, to the use of a transparent patch impregnated with certain chemical reactants which turn visible in the presence of water to show the location and intensity of sweating on various parts of the skin, and most specifically, these reactants being starch and iodine.

The Minor test is an iodine solution, often a USP iodine tincture, which is applied to an area of interest and allowed to dry. Subsequently, the area includes a starch. Sweating is then encouraged or allowed to occur naturally. When sweat reaches the surface of the skin, the presence of liquid allows the iodine and starch to combine, causing a color change to a very visible purple/black. This allows sweat production (intensity and specific location) to be easily seen.

The Minor test is used frequently in medical/drug research pertaining to sweating, in medical/drug research pertaining to conditions that alter sweating, and by medical professionals administering localized sweating treatments. The Minor test is ubiquitous and there is no commonly-accepted method for visual identification of sweating location and rate that has supplanted this test.

The mechanism of this Minor test is that when the amylose present in starch combines with polyiodide ions, they form a charge transfer complex which absorbs certain wavelengths of light, producing a dark purple look. The polyiodide ions and starch will not spontaneously combine when not in physical molecular contact, e.g. when physically separated in a solid state/dry suspension. However, the polyiodide ions are soluble in sweat (among other things) and so introducing sweat turns the dry suspension of starch and polyiodide ions into a solution where the two reactants quickly combine and produce the visual effect.

The Minor test has numerous drawbacks all stemming from the fact that messy iodine solution needs to be swabbed across the target area, then allowed to dry, and then messy starch needs to be imprecisely dusted across the target area.

The Minor test is inconvenient, as it requires iodine to be messily spread across a target area, often requiring clean-up, and great care so that the iodine does not stain clothes, furniture, or other instruments. It also requires powder to be dusted across the target area, which is nearly impossible to contain, and demands that the target area be kept in a particular orientation with respect to gravity (e.g. the subject must lie on their side for an underarm test). Powder will almost inevitably cover clothes, furniture, the floor, and other equipment. These two applications require great concentration and a degree of skill, making self-administration of a Minor test very difficult in most cases.

The Minor test is non-standardized, as it is nearly impossible to apply a consistent amount of iodine or starch per square centimeter of skin, meaning that the visual results of the exact same sweat patterns will vary from test to test. This significantly limits the use of the Minor test in quantitative determination of sweating location and intensity, constraining it to be an assistive, qualitative tool at best.

The Minor test is staining, as the iodine solution applied to the skin stains it, and these stains are difficult to wash out.

The Minor test is slow, as the iodine solution must be painstakingly applied. Then the subject and person administering the test must both wait until the solution dries. Then, powder must be carefully dusted across the target area. The time spent in setting up a Minor test lowers the quality of results, as a substantial amount of sweat may have already accumulated on the skin before the starch is applied, even if skin was dried before the application of the iodine.

The Minor test is uneven, as it is nearly impossible to uniformly apply starch to the target area as it lands in discontinuous clumps, meaning that potential areas of sweating are missed while others are incorrectly over-highlighted by the test.

The present invention attempts to solve these problems as well as others.

### SUMMARY OF THE INVENTION

Provided herein are systems, methods and compositions for a Skin Patch for Determination of Sweating Location and Rate.

The methods, systems, and apparatuses are set forth in part in the description which follows, and in part will be obvious from the description, or can be learned by practice of the methods, apparatuses, and systems. The advantages of the methods, apparatuses, and systems will be realized and attained by means of the elements and combinations particularly pointed out in the appended claims. It is to be understood that both the foregoing general description and the following detailed description are exemplary and explanatory only and are not restrictive of the methods, apparatuses, and systems, as claimed.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

### BRIEF DESCRIPTION OF THE DRAWINGS

In the accompanying figures, like elements are identified by like reference numerals among the several preferred embodiments of the present invention.

FIG. 1A is a bottom view of the adhesive patch, according to one embodiment.

FIG. 1B is a top view of the adhesive patch, according to one embodiment.

FIG. 2A is a top view of the adhesive patch disposed on the arm of a subject showing sweating after being attached, according to one embodiment.

FIG. 2B is a top view of the adhesive patch disposed on the arm of a subject for 20 minutes, according to one embodiment.

FIG. 3A is a top view of the adhesive patch disposed on the arm of a subject showing sweating after being attached, according to one embodiment.

FIG. 3B is a top view of the adhesive patch disposed on the arm of a subject showing sweat glands, according to one embodiment.

FIG. 4A is a perspective view of the adhesive patch disposed on the under arm of a subject, according to one embodiment.

FIG. 4B is a perspective view of the adhesive patch disposed on the underarm of a subject showing sweat, according to one embodiment.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### DESCRIPTION OF THE EMBODIMENTS

Generally speaking, the adhesive skin patch comprises a skin-contacting surface and a test window covered in a thin layer of anhydrous iodine and anhydrous starch particles on a test window. When applied to dry skin, the test window includes a generally translucent and yellowish appearance. As the skin begins to sweat, the test window turns purplish/black where sweat appears, initially showing the localization of individual active sweat glands, and, as sweating continues, the test window showing larger purple/black patches where the sweating is most intense. The adhesive skin patch is useful for research into sweating and disorders of sweating, including medical professionals attempting to apply localized sweating treatments, and for consumers wanting to educate themselves on where their sweating is most intense and therefore where it may be most useful to apply antiperspirant.

The adhesive skin patch allows for the application of a precise distribution of iodine and starch in dry suspension in a rapid and clean matter, by placing an adhesive patch containing the iodine and starch suspension onto the surface of the skin. The preparation of such a patch is particularly difficult as the starch and iodine cannot at any point coexist in a solution until sweat is present, or they will otherwise irreversibly become dark purple. The adhesive skin patch include the starch and iodine are present in the test window and coexist in solution only when sweat is present.

In an alternative embodiment, the adhesive skin patch uses hydrochromic materials that are optically active chemicals that change color in response to moisture content. Hydrochromic materials often are in the forms of inks, dyes, coatings or vinyls. The ink generally is a composition comprising a pigment, a dye, a resin, and a solvent. Specific examples of the pigment include cationic surfactants, anionic surfactants, nonionic surfactants, amphoteric surfactants, polyester surfactants, and polyamine surfactants. Examples of the dye include benzofuran, lactone, fluorane, and aniline compounds. The resin composition may be a mixture of a ketonic compound and a cellulose compound. Examples of the solvent include alcohols such as ethanol, isopropanol, 2-propoxyethanol, and terpineol, ketones such as acetone, acetates such as butyl carbitol acetate, and ethoxyacetyl chloride.

In one embodiment, the adhesive patch is prepared in a controlled production environment, and in other embodiments, there are other precise ways such an adhesive patch could be prepared. In other embodiments, a larger patch may be used. In one embodiment, the adhesive patch shows that there is sweating occurring within the area of interest, and in other embodiments, the adhesive patch shows precisely where within the area of interest the sweating is occurring.

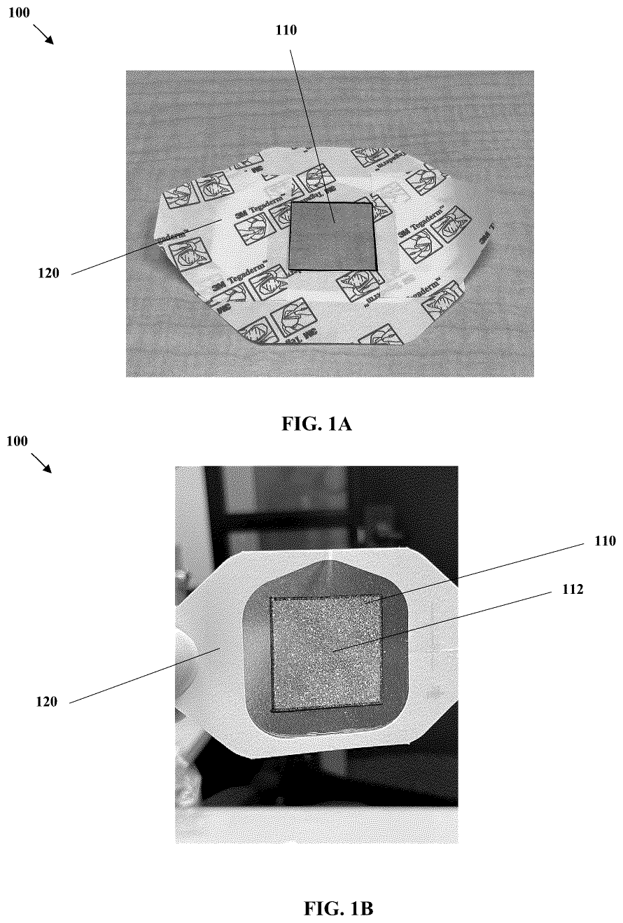

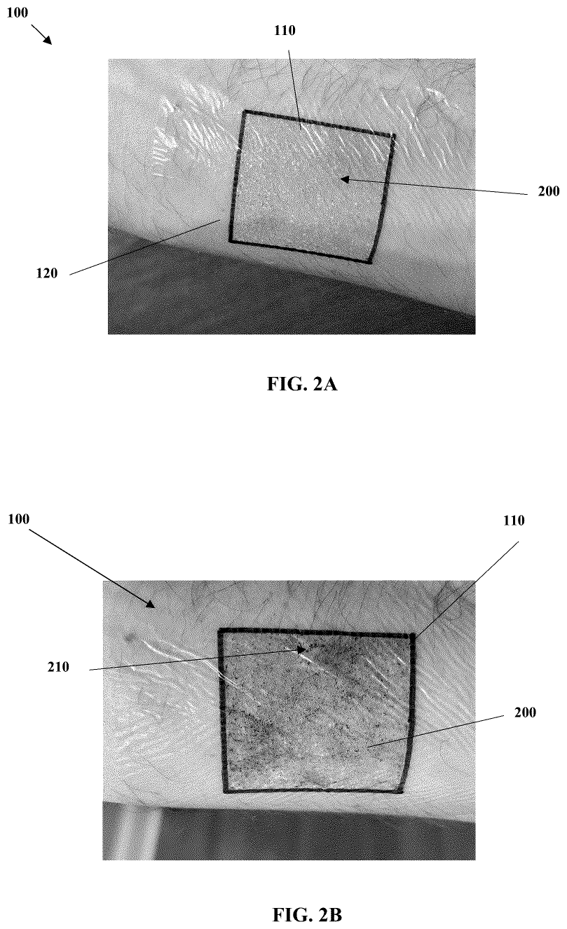

As shown in FIG. 1, the adhesive patch 100 includes the test window 110 is surrounded by an adhesive dressing 120. The test window 110 is transparent and includes a ˜1.5 sq. in. window in the center of it, applying ˜0.1 mL of USP Iodine Tincture to this test window and allowing the tincture to dry. After the Iodine tincture is dry, the test window is dusted with Azelis Corn PO4 PH “B” corn starch 112 using a powder pump to create an airborne dispersion of this powder that settled onto the window 110, as shown in FIG. 1B. This adhesive patch 100 may be applied to the skin or forearm and observed an almost immediate indication of slight sweating 200 at the location of some sweat glands, as shown in FIG. 2A. After twenty minutes, many more individual sweat glands 210 were visible, as well as a pattern of heavier sweating along part of the window test area 110, as shown in FIG. 2B.

In alternative embodiments, other carbohydrates may be used besides corn starch. Corn starch is a polymeric carbohydrate consisting of numerous glucose units joined by glycosidic bonds. Other polysaccharides include linear and helical amylose and the branched amylopectin. Depending on the plant, starch generally contains 20 to 25% amylose and 75 to 80% amylopectin by weight. Polysaccharides are hey are long-chain polymeric carbohydrates composed of monosaccharide units bound together by glycosidic linkages.

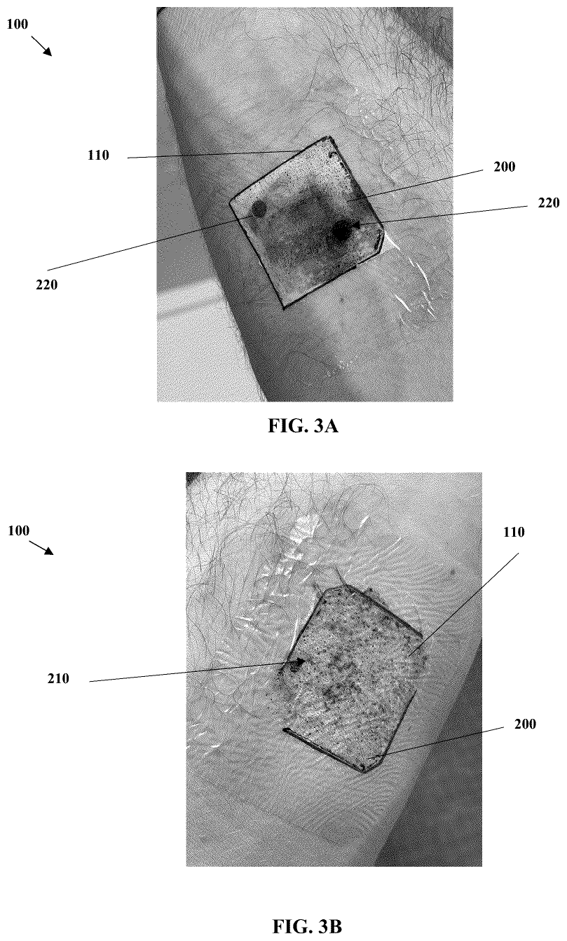

In other embodiments, alternative method of manufacturing of the adhesive patch are shown in FIGS. 3A-3B. Each patch 100 ultimately showing noticeable sweat patterns 200, and in some cases with “noise” introduced by manufacturing errors, such as the two dots 220 in FIG. 3A, where drops of iodine solution had been allowed to rest before being spread across the patch, leading to a relatively higher concentration of iodide ions than in other parts of the test window. Note the clear visibility of individual sweat glands in FIG. 3B, along with a region of heavier sweating across the center and bottom right of the patch.

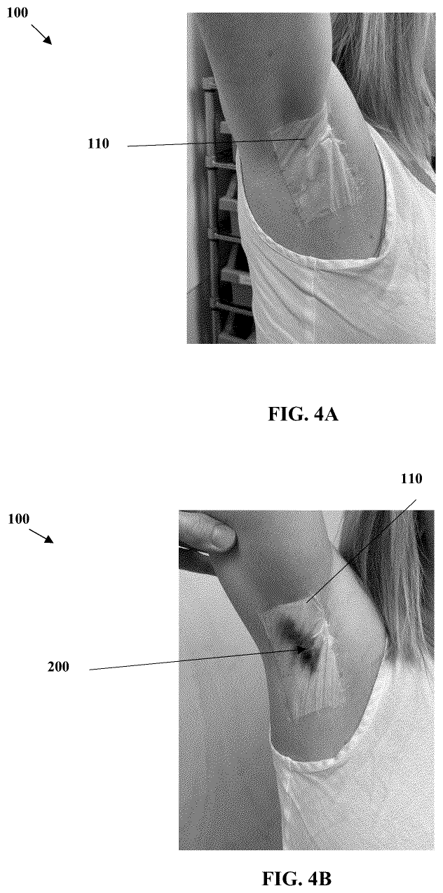

In another embodiment, the adhesive patch 100 is shown in FIGS. 4A-4B, where adhesive patch includes a size of about 2.5″×about 4″ sized for small underarms and show the specific utility of this patch size. The adhesive patch was manufactured via the same procedure as indicated above, utilizing 0.2 mL of USP Iodine Tincture and a dusting of Azelis Corn PO4 PH “B”. The adhesive patch was subsequently applied to a subject's towel-dried axilla. The test window 110 spans the entire surface of the adhesive patch, in this embodiment. After a wait time of five minutes, a clear pattern of axillary sweating 200 became visible on the patch, as shown in FIG. 4B. This adhesive patch demonstrates the small fraction of the patch sizes possible and the additional utility that arises from patches sized and shaped for particular body parts where phenomena such as sweating are desired to be examined, in this example case the axilla.

While the disclosed embodiments show the usable demonstration of the adhesive patch, the adhesive patch includes superior manufacturing methods that assure a precise and even distribution of both iodine and starch. In another embodiment, the test window includes adhesiveness across the test window when the adhesive is precisely allowed to comingle with the starch and iodine in dry suspension. In other embodiments, the shaping of adhesive patches mirrors various body parts, such as adhesive patches in a curved shape suited to the underarms and adhesive patches in a glove or sock shape suited for the hands and feet, respectively.

In one embodiment, the adhesive patch is convenient and can be applied in seconds by anyone, to nearly any body part (including self-application).

In one embodiment, the adhesive patch is Standardized. The amount of iodine and starch in the patch are always exactly the same, meaning the exact same sweating will produce the exact same results each time the invention is used. This allows for the invention's use in quantitative diagnostics and research, such as comparing the sweating of two separate subjects or the sweating of the same subject across time.

In one embodiment, the adhesive patch is non-staining. Iodine solution is never applied directly to the subject's skin—when the adhesive patches are removed, very little residue remained on the skin, which can be wiped off quickly and easily with a paper towel.

In one embodiment, the adhesive patch is rapid, where the adhesive patch can be applied in seconds, and the time it takes to conduct a sweat test is limited to the window during which sweating is to be observed. If the skin is dried immediately before application of an adhesive patch, the adhesive patch will indicate only sweat that has accumulated since the beginning of the adhesive patch application, rather than sweat that may have been accumulating while the test was being set up, as in the case of a traditional Minor test.

In one embodiment, the adhesive patch is even and the iodine and starch are uniformly distributed across the adhesive patch, ensuring that any differences in indication between one part of the adhesive patch and another part are caused solely by sweating. The uniform distribution of iodine and starch allows more precision in diagnostics and research.

In one embodiment, any consumer could quickly and easily see which parts of their armpit are actually producing the most sweat, and therefore what areas to be sure to cover when applying antiperspirant. This primary goal of consumers being able to quickly and conveniently administer the adhesive patch to themselves. Such consumer self-administration also has significant marketing benefits to companies providing these test patches, as the visual results are very conducive to sharing on the primarily visual mediums of consumer brand discovery and endorsement of our time

The core advantage, driving all the specific advantages of the adhesive patch, is that this replaces a skin test requiring the staged application of multiple reactants imprecisely across an area of skin, with a rapid and uniform way to precisely lay down all those reactants together.

As discussed above, the massively increased precision that the adhesive patch improves the convenience, standardization, and utility of the test for the medical professionals who employ it every day, resulting in better practicing and improved patient outcomes. Also as discussed above, the benefits of the adhesive patch to be used in more repeatable and predictable ways in more research pertaining to sweating or conditions that affect sweating.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. An adhesive patch for detecting sweat on a subject, comprising: an adhesive dressing surrounding a test window, wherein the test window is transparent;
the test window include a reactive chemical mixture which changes color in the presence of moisture; and
the test window changes color from transparent to indicate sweating.

2. The adhesive patch of claim 1, wherein the test window includes a surface of about 1.5 sq. in.

3. The adhesive patch of claim 1, wherein the reactive chemical mixture is selected from the group consisting of: iodine, starch, or a hydrochomic material.

4. The adhesive patch of claim 3, wherein the iodine and starch is generally translucent and yellowish appearance and turns purplish/black where sweat appears.

5. The adhesive patch of claim 4, wherein the iodine and starch do not mix while disposed on the test window and the iodine and starch coexist only when sweat is present in the test window.

6. The adhesive patch of claim 3, wherein the hydrochromic material is an optically active chemical that change color in response to moisture content, wherein the hydrochromic material comprises a pigment, a dye, a resin, and a solvent.

7. The adhesive patch of claim 6, wherein the pigment includes cationic surfactants, anionic surfactants, nonionic surfactants, amphoteric surfactants, polyester surfactants, or polyamine surfactants; the dye includes benzofuran, lactone, fluorane, or aniline compounds; the resin includes a mixture of a ketonic compound and a cellulose compound;
and the solvent includes alcohols, ketones, acetates, or ethoxyacetyl chloride.

8. A method of making an adhesive patch for detecting sweating of a subject, comprising: applying an iodine tincture to a transparent window surrounded by an adhesive dressing; allowing the iodine tincture to dry; dusting the dried iodine tincture with corn starch through a powder pump to apply a layer of corn starch; and applying the adhesive patch to a subject to detect sweating on the test window.

9. The method of claim 8, wherein the amount of the iodine tincture is about 0.1 mL.

10. The method of claim 9, wherein the test window includes a surface of about 1.5 sq. in.

11. The method of claim 10, wherein the iodine and starch do not mix while disposed on the test window and the iodine and starch coexist only when sweat is present in the test window.

12. A method of making an adhesive patch for detecting sweating of a subject, comprising: applying a hydrochromic ink or pigment to a transparent window surrounded by an adhesive dressing; allowing the material to dry; and applying the adhesive patch to a subject to detect sweating on the test window.

13. The method of claim 12, wherein the test window includes a surface of about 1.5 sq. in.
